(19) United States
(12) Patent Application Publication
(10) Pub. No.: US 2004/0115767 A1
(43) Pub. Date: Jun. 17, 2004

(54) Immunogenic cell surface proteins of helicobacter pylori

(76) Inventors: Asa Ljungh, Lund (SE); Ingrid Nilsson, Lund (SE); Meeme Utt, Lund (SE); Torkel Wadstrom, Lund (SE)

Correspondence Address: Bacon & Thomas, 4th Floor, 625 Slaters Lane, Alexandria, VA, 22314-1176, US

(21) Appl. No.: 10/471,594

(22) PCT Filed: Mar. 20, 2002
(86) PCT No.: PCT/SE02/00535

(30) Foreign Application Priority Data
Mar. 23, 2001 (SE) 0101030-5

(51) Int. Cl. C12P 21/06

(52) U.S. Cl. 435/69.1

(57) ABSTRACT

A method of identifying immunogenic Helicobacter pylori-specific surface proteins binding specifically to polysulphated molecules, is described. Further, a Helicobacter pylori-specific surface protein product, and a protein contained therein, binding specifically to polysulphated molecules are disclosed. Examples of such proteins are a protein having a MW of 30 (31.3) kDa, a (pI) of 9.3 (9.1) and comprising SEQ ID NO: 1 and/or SEQ ID NO: 2; a protein having a MW of 26 (27.7), a pI of 9.1-9.3 (9.0) and comprising SEQ ID NO: 4; or a protein having a MW of 25 (28.7), a pI of 9.0 (8.8) and comprising SEQ ID NO: 5. Additionally, use of a protein or protein product of the invention as a diagnostic antigen and as a component in a vaccine against H. pylori infection, as well as an immunoassay and a vaccine composition against a H. pylori infection, are described.

The present invention relates to immunogenic cell surface proteins of Helicobacter pylori applications of the proteins and a method of producing them.

### BACKGROUND

Helicobacter pylori is a spiral shaped microorganism colonising the human gastric epithelium inducing type B gastritis and peptic ulcerations (Marshall, 1994). A diagnostic test for detecting this infection should be reliable, cost-effective, easy to perform and preferably non-invasive. In most H. pylori infected individuals there is a strong but highly polymorphic humoral immune response to a number of antigens, and detection of antibodies to various antigens has widely been used for serodiagnosis (Bazillou et al., 1994; De Boer, 1997; Aucher et al. 1998). Such serological assays have been based mainly on enzyme immuno assays (EIA) and immunoblot assays using various antigen preparations. Several available commercial kits use antigen preparations whose compositions are unknown to the user, even though the performance of EIAs depends on the protein composition used in such an assay (Feldman et al., 1995; Meijer et al., 1997). Some antigens of H. pylori such as heat shock proteins (Hsps), flagella and urease cross-react with antigens from other bacterial species, which makes it difficult to choose rationally the antigenic extract or extraction method.

In a previous study it was found (Nilsson et al., 1997) that antibody reactivity to a set of low molecular mass (Mᵣ) cell surface released proteins (25 to 35 kDa) correlated strongly with H. pylori infection. However, several of these proteins were not satisfactorily resolved or co-migrated in the one-dimensional electrophoresis (1-DE). However, the use of two dimensional electrophoresis (2-DE) techniques allows a higher resolution of proteins, in which the appropriate pH gradient is of great importance (Kimmel et al., 2000). In a recent 2-DE study (Nilsson et al.; 2000) a strong immune response to H. pylori proteins of Mᵣs 25-30 kDa was noted. However, these proteins remained unresolved by 2-DE in the pH range of 3-10.

### DESCRIPTION OF THE INVENTION

The present invention combines chromatographic enrichment of low abundant proteins of H. pylori with 2-DE using a narrow pH gradient for separation of co-migrating polypeptides, followed by mass spectroscopic identification of proteins. Further, the usefulness of two-dimensional (2-D) immunoblot for identification of immunogenic and cross-reactive proteins is demonstrated.

The invention is in one aspect directed to a method of identifying immunogenic Helicobacter pylori-specific surface proteins binding specifically to polysulphated molecules, e.g. glucosaminoglycans and/or other sulphated glycoconjugates such as mucins, comprising the steps of

cultivating H. pylori bacteria in vitro,

isolating the cultivated bacteria,

releasing the basic surface proteins by acid glycine extraction,

removing the bacteria, and

purifying the glycine extract to produce a Helicobacter pylori-specific surface protein product, and

subjecting the protein product to a two-dimensional immunoblot to identify immunogenic proteins.

In an embodiment of the method according to this aspect of the invention,

the cultivating in vitro is performed in an agar medium or broth,

the isolation of the bacteria is performed by collecting the grown cells into phosphate a buffered saline (PBS) and centrifugation to produce a pellet,

releasing the basic surface proteins from the pellet by acid glycine extraction,

removing the bacteria by centrifugation, and

purifying the glycine extract by diluting the supernatant from the centrifugation with PBS, adjusting the pH to 6.5 and subjecting the mixture to heparin affinity chromatography to produce the Helicobacter pylori-specific surface protein product.

Another aspect of the invention is directed to a Helicobacter pylori-specific surface protein product binding specifically to polysulphated molecules, e.g. glucosaminoglycans and/or other sulphated glycoconjugates such as mucins, which product is obtainable by

cultivating H. pylori bacteria in vitro,

isolating the cultivated bacteria,

releasing the basic surface proteins by acid glycine extraction,

removing the bacteria, and

purifying the glycine extract to produce the Helicobacter pylori-specific surface protein product.

In an embodiment of this aspect of the invention the protein product is obtainable by that

the cultivating in vitro is performed in an agar medium or broth,

the isolation of the bacteria is performed by collecting the grown cells into a phosphate buffered saline (PBS) and centrifugation to produce a pellet,

releasing the basic surface proteins from the pellet by acid glycine extraction,

removing the bacteria by centrifugation, and

purifying the glycine extract by diluting the supernatant from the centrifugation with PBS, adjusting the pH to 6.5 and subjecting the mixture to heparin affinity chromatography to produce the Helicobacter pylori-specific surface protein product.

In a presently preferred embodiment the product comprises at least one protein selected from the group consisting of

a) a protein having a molecular weight (MW) of 30 (31.3) kDa, an isoelectric point (pI) of 9.3 (9.1) and comprising the amino acid sequence SEQ ID NO: 1 and/or SEQ ID NO: 2,

b) a protein having a MW of 26 (27.7), a pI of 9.1-9.3 (9.0), and comprising the amino acid sequence SEQ ID NO: 4, and

c) a protein having a MW of 25 (28.7); a pI of 9.0 (8.8) and comprising the amino acid sequence SEQ ID NO: 5.

Yet another aspect of the invention is directed to a Helicobacter pylori-specific surface protein binding specifically to polysulphated molecules and having a MW of 30 (31.3) kDa, a (pI) of 9.3 (9. 1) and comprising the amino acid sequence SEQ ID NO: 1 and/or SEQ ID NO: 2; a Helicobacter pylori-specific surface protein binding specifically to polysulphated molecules and having a MW of 26 (27.7), a pI of 9.1-9.3 (9.0) and comprising the amino acid sequence SEQ D NO: 4; or a Helicobacter pylori-specific surface protein binding specifically to polysulphated molecules and having a MW of 25 (28.7), a pI of 9.0 (8.8) and comprising the amino acid sequence SEQ ID NO: 5.

Still another aspect of the invention is directed to the use of a protein product according to the invention or a protein according to the invention as a diagnostic antigen in an immunoassay.

A further aspect of the invention is directed to the use of a protein product according to the invention or a protein according to the invention as an immunizing component in a vaccine against H. pylori infection.

An additional aspect of the invention is directed to an immunoassay for the determination of the presence of H. pylori bacteria in a biological sample from a human patient, wherein a protein product according to the invention or a protein according is used as a diagnostic antigen. Any immunoassay based on antigen-antibody interaction may be used, such as enzyme linked. immunosorbent assay (ELISA), radioimmunoassay (RIA) etc.

A final aspect of the invention is directed to a vaccine composition against a H. pylori infection in a human patient comprising as an immunizing component at least one protein product according to the invention or a protein according to the invention, together with a pharmaceutically acceptable vehicle. The pharmaceutically acceptable vehicle will be selected by the manufacturer, e.g. with the guidance of the US or European pharmacopoeia.

The invention will now be illustrated by description of experiments and the drawings, but it should be understood the invention is not limited to any specifically disclosed example.

Experiments

Material and Methods

A. Bacterial Strain and Culture Conditions

H. pylori strain CCUG 17874 was cultured from a frozen stock (−115° C.) on GAB-CAMP agar plates (Soltesz et al., 1992) for three days at 37° C. in a microaerophilic atmosphere (5% O₂, 10% CO₂, 85% N₂). Cells were harvested, washed once in phosphate buffered saline pH 7.2 (PBS, 0.02 M sodium phosphate, 0.15 M NaCl) and kept for subsequent protein extraction.

B. Extraction of Cell Surface Proteins

Acid glycine extraction of cell surface proteins from 3-day old cells was performed as previously described (Lelwala-Guruge et al., 1990). In brief, harvested and washed calls were re-suspended in 0.2 M glycine hydrochloride, pH 2.2 (4 g cells/100 ml) and stirred magnetically for 15 min at 20° C. supplemented with protease inhibitors (Complete™, Roche Diagnostics AB, Bromma, Sweden). Cells were removed by centrifugation (12,000×g for 15 min at 8° C.) and the supernatant neutralised with NaOH and dialysed for 18 h at 8° C. against PBS. Protein was quantified to 210 μg/ml by the Bradford method using the BioRad protein assay BioRad, Richmond, Calif. U.S.A.) and bovine serum albumin as a standard. This protein fraction is further referred to as AGE-proteins.

C. Heparin Affinity Chromatography

A HiTrap heparin column (one ml, Amersham Pharmacia Biotech, Uppsala, Sweden) was equilibrated with 5 mM sodium phosphate buffer and 75 mM NaCl, pH 6.5. AGE-proteins were diluted with the same buffer 1:3, pH adjusted to 6.5 and applied to the affinity column. Unbound proteins were washed out with the equilibration buffer, whereas bound proteins eluted in a single peak using 2 M NaCl; yielding 656 μg/ml in a 2.5 ml protein fraction, hereafter referred to as AGE-HepBP (acid glycine extracted-heparin binding proteins). The AGE-HepBP fraction was desalted on a Fast Desaltng® column HR 10/10 (Amersham Pharmacia Biotech) at a flow rate of 1 ml/min, equilibrated with 8 M urea (Merck Eurolab AB, Stockholm, Sweden) in water, aliquoted and kept frozen at −20° C.

D. One-dimensional Sodium Dodecyl Sulphate-polyacrylamide Gel Electrophoresis (1-DE) and Immunoblot (1-D immunoblot)

1-DE was performed under reducing conditions (Laemmli, 1970) using Criterion™ Cell electrophoresis equipment (BioRad, Richmond, Calif., U.S.A.) with an 8-16% separating gradient gel and a 4% stacking gel (Criterion Precast Gel, BioRad). Proteins were diluted in a standard sample buffer, loaded to the gel (75 μg/gel) and separated at 200 V for 80 min. Mᵣs of the separated AGE-HepBP were established using Precision marker proteins (BioRad). Following electrophoresis, gels were fixed and silver stained (Heukeshoven et al., 1988). Alternatively, proteins were transferred to a polyvinylidene difluoride membrane (PVDF) (Micron Separations Inc. Westborough, Mass., U.S.A.) for antibody detection, using a semi-dry electro-blotter equipment (Ancos, Vig, Denmark). Transfer time was 80 min at a constant current of 1 mA/cm² The immunoblot assay was performed as previously described (Nilsson et al., 1997). Briefly, the membranes were blocked for 2×15 min in buffers containing hydrolysed gelatine, polyvinyl-pyrolidone, Tween 20, ethanolamine and glycine, cut into strips and probed with a panel of sera diluted 1/100 in a buffer containing TRIS, gelatin hydrolysate, sodium chloride, Tween 20, pH 8.7 (Nilsson et al., 1997). Sera were obtained from patients with various diseases including; i) H. pylori infection (n=10) established by positive culture of endoscopic biopsy specimen and serology; ii) a pool of sera from H. pylori culture negative patients; iii) rheumatoid arthritis (n=8) presenting with high rheumatoid factor titres by the Waaler-Rose test; iv) Campylobacter jejuni infection (n=5) presenting with high antibody titres, v) cystic fibrosis (n=3) with Pseudomonas aeruginosa colonisation in the lungs; vi) Treponema pallidum infection (n=3) positive by the TPHA test; and vii) Haemophilus influenzae infection (n=3) positive by culture. All serum samples were collected at the Department of Clinical Microbiology, Lund University Hospital, Lund and kept frozen at −20° C. until tested.

E. Isoelectric Focusing (IEF)

The AGE-HepBP were resolved by IEF on precast Immobiline® Dry strips with a linear pH gradient, pH 6-11, 11 cm (IPG strips, Amersham Pharmacia Biotech). The IPG strips were re-hydrated, according to the manufacturer's instructions, for 12 h at 20° C. together with the AGE-HepBP (approx. 25 μg per strip) in a 200 μL solution containing 8 M urea, 3-(3-chloroamidopropyl)dimethylammonio-1-propane sulphonate (CHAPS) (20g/L), IPG sample buffer pH 6-11 (Amersham Pharmacia Biotech), dithiotreitol (DTT) (10 g/L) and 25 μL bromphenol blue (0.5% w/v). IEF was performed in an IPGphor focusing apparatus (Amersham Pharmacia Biotech) for a total of 80 kVh.

F. Two-dimensional Polyacrylamide Gel Electrophoresis (2-DE)

Before a 2-DE was started, IPG-strips were equilibrated for two intervals of 20 min each in a solution containing 50 mM Tris-HCl, pH 8.8, 6 M urea, 30% (v/v) glycerol, and 1% (wt/v)sodium dodecylsulphate (SDS). DTT (1%, w/v) was added to the first equilibration solution and iodacetamide (4.8%, w/v) was added to the second equilibration solution. IPG-strips were then applied on the top of 8-16% gradient SDS-PAGE Criterion Precast Gels (BioRad) and separation of proteins carried out under the same conditions as described for 1-D SDS-PAGE. Gels were then processed for silver staining, Coomassie R-350 (PhastGel® Blue R, Amersham Pharmacia Biotech) and for transfer of proteins to PVDF membranes. The IEF and the 2-DE experiments were repeated three to four times.

G. 2-D Immunoblot Assay

Polypeptides resolved by 2-DE were electrophoretically transferred to PVDF membranes as described in section D and residual binding capacity was blocked as described before. Membranes were placed in polyethylene bags and overlaid with; i) pooled serum samples (n=10) obtained from patients, referred for gastroscopy due to abdominal pains, and positive for H. pylori by culture of gastric biopsy; ii) pooled serum samples (n=10) from H. pylori culture and sero-negative persons; iii) serum from a H. pylori infected child, positive by culture of gastric biopsy, and iv) serum from a C. jejuni infected patient. Sera were diluted 1/100 or 1/1000 in 10 ml incubation buffer (see section D.) and incubation time was 16 b at 8° C. under constant shaking. After repeated washings, horseradish peroxidase-labelled anti-human IgG antibodies (Dako A/S, Glostrup, Denmark) diluted 1/500 were added and the membrane was then incubated for 2 h at 20° C. Bound antibodies were detected by adding a 50 mM sodium acetate buffer (pH 5.0) containing 0.04% 3-amino-9-ethylcarbazole (Sigma Chemicals Co., St. Louis, Mo.) and 0.015% H₂O₂.

H. Image Analysis

Gels and immunoblots were scanned with a GS-710 Imaging Densitometer (BioRad), 1-DE gel images were analysed and Mᵣs of proteins were estimated using the Quantity One software package (BioRad). The 2-DE gel image analysis, including estimation of pI and Mᵣs, spot quantification, and matching between gels and 2-D immunoblots was achieved with the Melanie version 3 software (BioRad).

I. Spot Identification

Six spots, showing distinct antibody reactivity with the H. pylori positive serum pool, were selected for MS/MS sequencing, cut out from the gel under clean conditions and fragmented with trypsine. The peptide mixture was extracted and analysed by MALDI-TOF MS/MS. Obtained internal peptide sequences were used to identify proteins of the H. pylori genome at TIGR database (http://www.tigr.org/).

### DESCRIPTION OF THE DRAWINGS

FIG. 1. Protein band pattern of the AGE-HepBPs of H. pylori separated by 1 DE (8-16% gradient gel) and stained by Coomassie R-350. Protein standards are shown on left side.

FIG. 2. Antibody reactivity to the AGE-HepBP fraction. Strips were probed with sera from H. pylori infected and non-infected patients. Lane 1: H. pylori positive serum pool; lane 2: H. pylori negative serum pool; lanes 3-9: H. pylori culture positive patients; lanes 10-11: P. aeruginosa infected patients; lanes 12-13 C. jejuni infected patients; lanes 14-15 sera from rheumatoid arthritis patients; lanes 16-17: T. pallidum infected patients. Serum dilution 1/100. Protein standards are shown on left side.

FIG. 3A. AGE-HepBP of H. pylori resolved by IEF in the pH range 6-11 and with a Criterion 8-16% gradient gel stained by silver. Numbers 1-6 corresponds to the identified proteins in Table 1. Circle shows spots not recognised with the H. pylori positive serum pool (see circle FIG. 3C). Protein standards are shown on the right side.

FIG. 3B. Coomassie R-350 staining of AGE-HepBP of H. pylori resolved by IEF in the pH range 6-11 and with a Criterion 8-16% gradient gel. Numbers 1-6 corresponds to the identified proteins in Table 1. Protein standards are shown on the right side.

FIG. 3C. 2-D immunoblot with the AGE-HepBP faction of H. pylori resolved by IEF in the pH range 6-11 and with a criterion 8-16% gradient gel. The membrane was probed with a pool of 10 sera from H. pylori infected patients (dilution 1/1000). Numbers 1-6 corresponds to the identified proteins in Table 1. Protein standards are shown on the right side.

FIG. 4. 2-D immunoblot with the AGE-HepBP fraction of H. pylori resolved by IEF in the pH range 6-11 and with a Criterion 8-16% gradient gel. The membrane was probed with a pool of 10 sera from H. pylori negative patients (dilution of 1/100). Number 6 corresponds to protein in Table 1. Protein standards are shown on the right side.

FIG. 5. 2-D immunoblot with the AGE-HepBP fraction of H. pylori resolved by IEF in the pH range 6-11 and with a Criterion 8-16% gradient gel. The membrane was probed with a serum from a C. jejuni infected patient (dilution of 1/100) Number 6 corresponds to protein in Table 1. Protein standards are shown on the right side.

### RESULTS

AGE-HepBP Protein Profile in 1-DE and 1-D Immunoblot

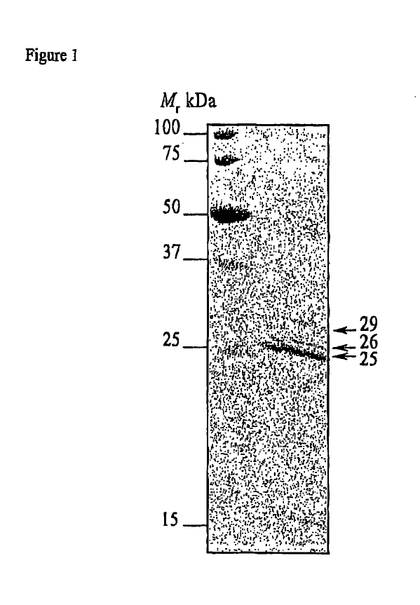

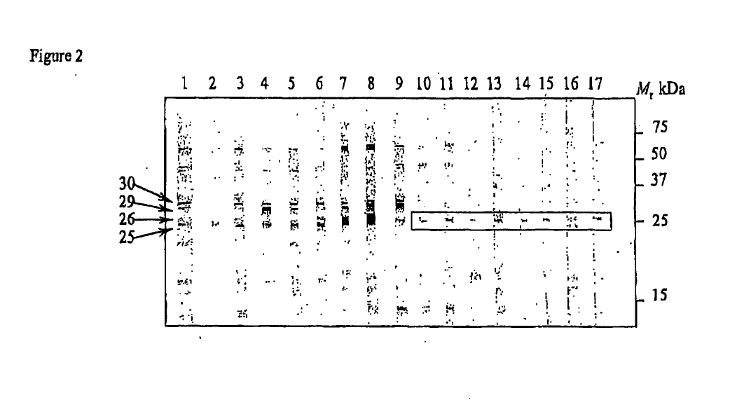

The protein profile of the AGE-HepBP, separated by 1-DE and stained by Coomassie R-350 is shown in FIG. 1. Three bands were identified with Mᵣs of 25, 26 and 29 kDa. Antibody reactivity to four bands (Mᵣs 25, 26, 29 30 kDa) was detected with sera of H. pylori infected patients and also by the child patient. Differences in intensity of these bands probably depend from an individual immune response among the patients (FIG. 2, lanes 1, 3-9). The H pylori seronegative serum pool showed weak reactivity to a 25 kDa and 26 kDa protein (FIG. 2, lane 2). Other potentially cross-reactive sera demonstrated reactivity to a 25 kDa protein (FIG. 2, lanes 10-17, in frame). Reactivity to proteins of Mᵣs >37 kDa were not estimated in the present study.

AGE-HepBP Protein Profiles in 2-DE and 2-D Immunoblot

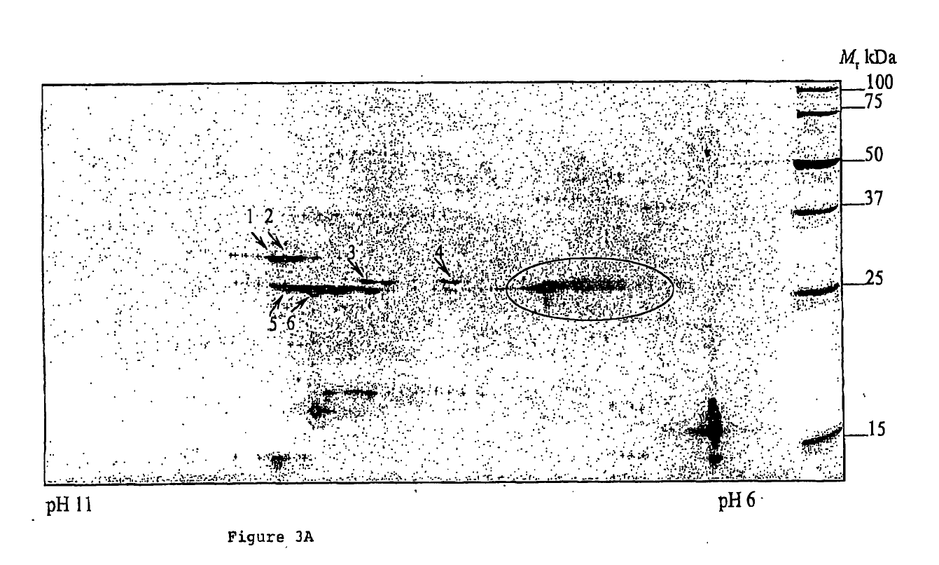

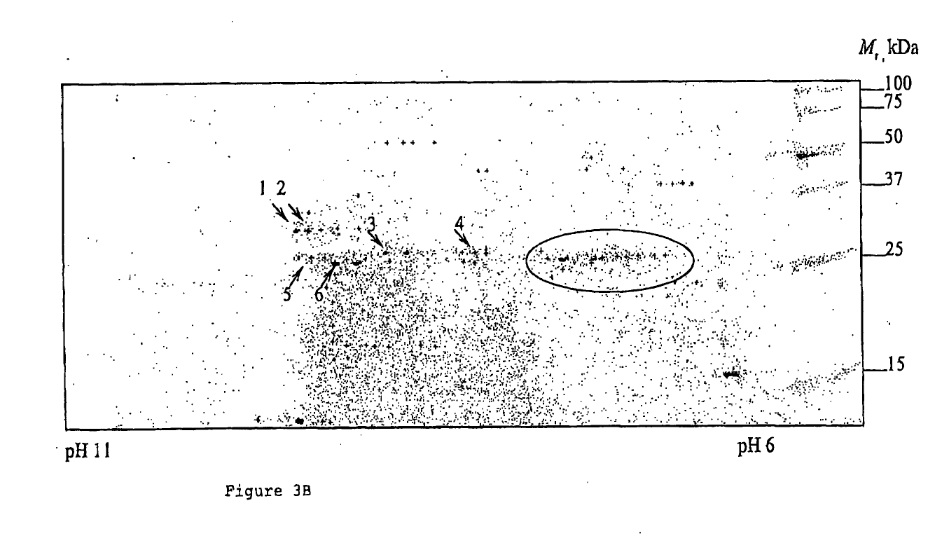

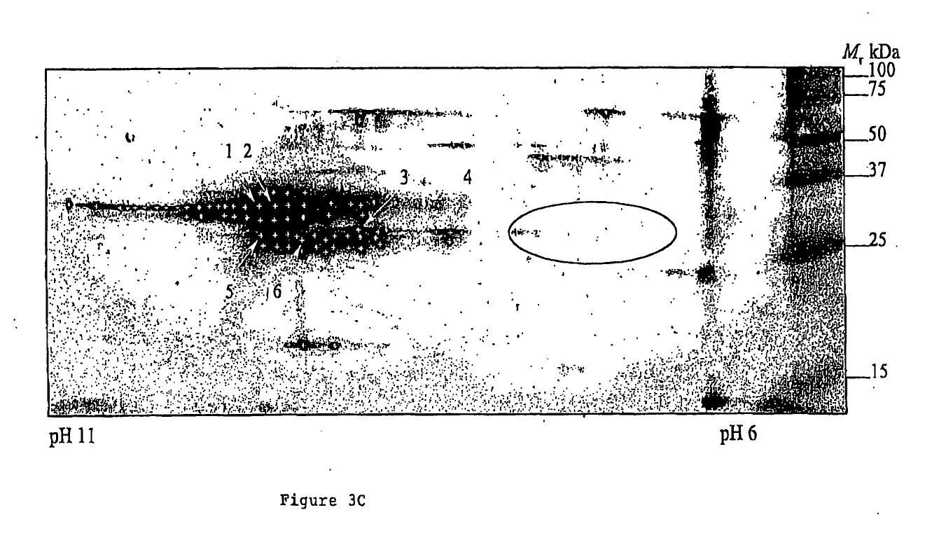

A total of 141 spots were detected on silver stained gels (FIG. 3A) in comparison to 71 spots detected on Coomassie R-350 stained gels (FIG. 3B). Using 2-D immunoblot, 186 spots showed antibody reactivity with the H. pylori positive serum pool with a dilution 1/1000 (FIG. 3C). A higher number of spots were detected by immunoblot demonstrating the increased sensitivity of antibody detection compared with silver and Coomassie staining of spots. Repeated 2-DE experiments demonstrated a high reproducibility with the difference of estimated pI values varying between 0.0 to 0.05 units. No variability in estimation of Mᵣs was observed. Some spots that stained strongly using silver and Coomassie stains (FIGS. 3A and B, circle) were not immunogenic (FIG. 3C). The pI and Mᵣ values obtained using the Melanie 3 software are listed in Table 1.

Identification of Spots (Proteins)

Internal peptide sequences of six spots (FIGS. 3A-C, spots no 1-6, Table 1) were identified as peptides of cell binding factor 2 (spots no 1 and 2), the urease A subunit (spots no 3 and 4), a hypothetical protein (spot no 5) and an outer membrane protein (spot no 6).

Cell binding factor 2 (HP0175) has a similarity to cell-binding factor 2 of C jejuni (antigen PEB4A) and is a homologue to Escherichia coli survival protein surA. Here two were identified, but a further two-three isoforms with lower pI's could be proposed with the same Mᵣs. Cross reactivity with serum from a C. jejuni infected patient was not observed.

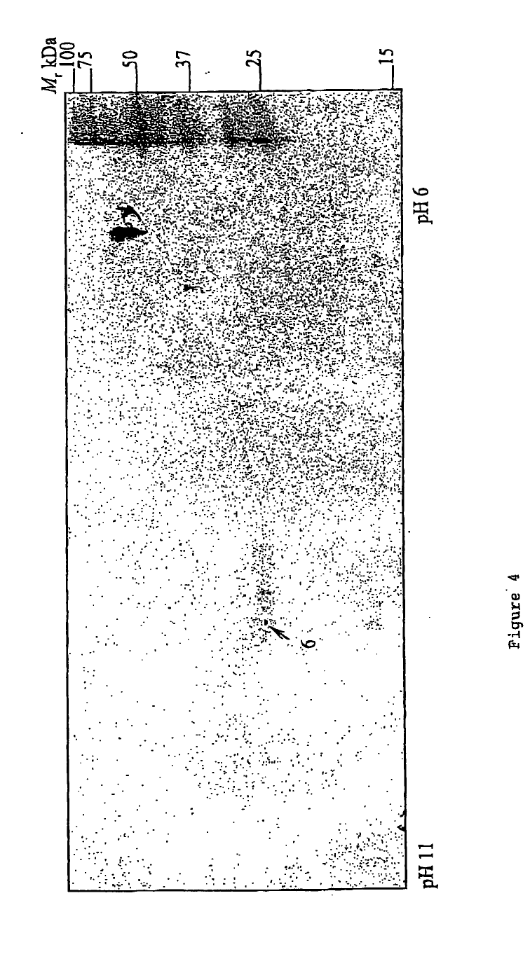

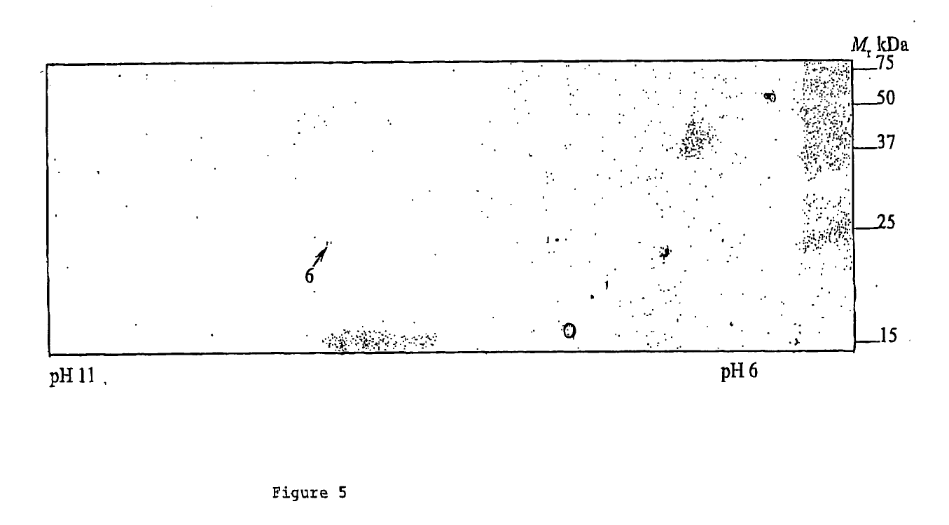

Spot number 5 (HP0231) was identified as a hypothetical protein with unknown function. This protein co-migrates in 1-DE with the urease. A subunit and outer membrane protein HP 1564, but was separated by 2-DE. It was recognised by the H. pylori positive serum pool with a dilution 1/1000 by 1-D immunoblot, whereas no reactivity with the H. pylori negative serum pool (FIG. 4) or C. jejuni serum was observed (FIG. 5).

The outer membrane protein identified (HP1564) belongs to the lipoprotein-28 super family and shows similarity to a Pasturella haemolytica lipoprotein 1, (probably attached to the outer membrane by a lipid anchor) and is also similar to both an E. coli hypothetical 29.4 kDa lipoprotein and to a 28 kDa Haemophilus influenzae lipoprotein (hlpA, outer membrane protein HI0620, 38.5% identity by BLAST software). This protein (FIG. 3A-C, spot no 6, the 25 kDa band in FIG. 1) showed reactivity with all potentially cross reactive sera used in this study and with the H. pylori negative serum pool (FIG. 2).

Utilization of Results

Defining surface proteins of the human gastric pathogen H. pylori is of great interest as several of these proteins are candidates to optimise immunodiagnostic tests and for vaccine design A large number of antibody screening tests have been developed based on poorly defined H. pylori antigens (Glupczynski. 1998). The 2-DE technique facilitates identification of proteins in a complex antigen to a higher degree than does 1-DE and to overcome the problem of co-migrating proteins when 1-DE is used. As shown in this study, proteins separated as a 1-DE band were separated by the 2-DE technique into several distinct protein spots by using narrow pH gradients.

The 2-DE in combination with immunoblot has previously been described for identification of H. pylori antigens (McAtee et al., 1998b; Jungblut et al., 2000). Jungblut with coworkers used a single patient serum on 2-D immunoblot, where whole cell proteins were resolved and antibody reactivity to a relatively small number of immunogenic proteins was found. These results suggest that several antigens might be minor components in whole cell lysates and therefore difficult to determine by immunoblot. The individual host response may also play a role.

Chromatographic fractionation and enrichment helps overcome this problem. Using a heparin affinity chromatography fractionation before 2-DE, it was possible to enrich certain low abundant proteins and we were able to show for the first time the binding of hypothetical protein HP023 1 to heparin and its immunogenic property. Chromatography has been used in combination with 2-DE for enrichment of minor proteins of H. influenzae and using heparin affinity chromatography, 110 new proteins were identified (Fountoulakis et al., 1997). In another study enrichment of low abundant human brain proteins was performed with this technique Karlsson et al, 1999). Hydroxyapatite, another chromatographic matrix was used to enrich low abundant proteins ofE. coli (Fountoulakis et al., 1999).

Broad pH gradients have earlier been used to separate H. pylori proteins (McAtee et al., 1998b; Jungblut et al. 2000; Nilsson et al., 2000). In these studies basic proteins were resolved to a minor degree. However, in the present study we obtained a higher resolution of basic proteins by using a narrow pH 6-11 gradient in the IEF. This pH gradient is important in studies of H. pylori proteins since more than 70% of the coded precursor proteins have a theoretical pI higher than 7 (Tomb et al., 1997).

Antibodies raised against antigens of low Mᵣ were found to be of prognostic value for H. pylori infection where, antibody reactivity to a 33-35 kDa antigen was present in 97.5% of patients with gastric or duodenal ulcer but less often in patients with chronic type B gastritis (Yamaoka et al., 1998). An antibody response to a 26 kDa protein (HP 1563, alkyl hydroperoxide reductase tsaA) was found in sera from gastric cancer patients but not in sera from non-cancer H. pylori infected patients (Wang et al., 1998). In a previous immunoblot study, antibody reactivity to a set of low Mᵣ surface proteins (26 to 33 kDa) was found to strongly correlate with H. pylori infection (Nilsson et al., 1997). Antibody reactivity to the proteins in this range were also found in this study and three novel antigens were identified.

Cell binding factor 2 as an immunogenic protein of H. pylori was recently described (McAtee et al., 1998a; Jungblut et al., 2000) but no data concerning cross reactivity was reported. Since this protein is similar to the C. jejuni cell binding protein 2, we examined potential cross reactivity to this protein with serum of a C. jejuni infected patient and with other potentially cross reactive sera. No antibody reactivity was found even at a low dilution of sera (1/100). The H. pylori-positive serum pool gave a distinct staining at a tenfold higher dilution (1/1000) (FIG. 5 and 4, respectively) Thus, this protein demonstrated a strong, immunogenicity and high specificity. Since the signal peptide in precursor sequence could be predicted and a mild protein extraction method releasing mainly surface exposed proteins was used, it is reasonable to believe that this protein is located on the cell surface. Showing a strong immunogenicity low cross reactivity and cell surface exposure makes this protein a (cell binding factor 2) appropriate as candidate for diagnostic purpose and vaccine development.

The hypothetical protein HP0231 demonstrated a heparin binding function and together with other surface exposed heparin binding proteins it may be involved in binding of H. pylori to cell surface and matrix associated glycosaminoglycans (GAG). This may lead subsequently to the binding of host proteins (Duensing et al., 1999) and H. pylori cells coated with host proteins may escape from the attack of host defense system. It may also increase the ability of bacteria to adhere to the acidic fraction of mucin, cell surface exposed glycosaminoglycans and extracellular matrix components so as heparin sulphate without their own specific receptors to maintaining continuous colonisation of this pathogen.

The outer membrane protein HP1564 (lipoprotein 28) and urease A subunit co-migrate in 1-DE, but were resolved with 2-DE. Both proteins are immunogenic, however the HP1564 was also recognised by a set of cross reactive sera and the H. pylori negative serum pool. The urease of H. pylori is known to have similarity with ureases from other species and the weak staining of the 25-26 kDa protein by 1 D immunoblot could lead to a false positive result. Thus, only a distinct antibody reactivity could be interpreted as a H. pylori positive signal.

Except for the urease A, we predicted a signal sequence for all three identified proteins using the signal P software at http://www.cbs.dtu.dk/services/SignalP/. This is consistent with our earlier observation, that these proteins are surface exposed on H. pylori and therefore would be useful for diagnostics and vaccine development.

Interestingly, several abundant proteins in our protein mixture were not immunogenic. (FIGS. 3 A-C, circle). This demonstrates that a complex antigen may contain proteins not recognised by patient sera and these proteins may have a reducing effect on the concentration of the immunogenic proteins causing a reduction of effective coating of ELISA plates or protein load for immunoblots.

In conclusion, sample fractionation and enrichment of proteins using a chromatographic step prior to 2-DE improves the possibility to identify proteins present at low concentration. It also may improve the ratio of immunogenic vs non-immunogenic proteins in antigen preparation Using the 2-D immunoblot, we identified two new immunogenic H. pylori proteins, i.e. cell binding factor 2 (HP0175 ) and a heparin binding protein (HP0231) which may be used in serodiagnostic tests and for vaccine development. We found that 2-D immunoblot also helps to identify cross reactive proteins in complex antigens used in diagnostic tests. In the post-genomic era, when hundreds of microbial genomes will be available, more precise identification of immunogenic proteins will be necessary, either using classical N-terminal microsequencing or more advanced MS/MS sequencing.

The present method will allow characterisation of antigenic proteins for further improvement of specificity in serological tests.

### REFERENCES

Aucher, P., Petit, M. L., Mannant, P. R., Pezennec, L., Babin, P., Fauchere, J. L., 1998. Use of immunoblot assay to define serum antibody patterns associated with Helicobacter pylori infection and with H. pylori related ulcers. J. Clin. Microbiol. 36, 931-936.

Bazillou, M., Fendri, C., Castel, O., Ingrand, P., Fauchere, J. L. 1994. Serum antibody response to the superficial and released components of Helicobacter pylori. Clin. Diagn. Lab. Immunol. 1, 310-307.

De Boer, W. A., 1997Helicobacter pylori and non-ulcer dyspepsia. Scand. J. Gastroenterol. 32, 1183-1184.

Duensing, T. D., Wing, J. S., van Putten, J. P., 1999. Sulfated polysaccharide-directed recruitment of mammalian host proteins: a novel strategy in microbial pathogenesis. Infect. Immun. 67, 4463-4446.

Feldman, R. A., Deeks, J. J., Evans, S. J., 1995. Multi-laboratory comparison of eight commercially available Helicobacter pylori serology kits. Helicobacter pylori Serology Study Group. Eur. J. Clin. Microbiol. Infect. Dis. 14, 428A433.

Fountoulakis, M., Langen, H., Evers, S., Gray, C., Takacs, B., 1997. Two-dimensional map of Haemophilus influenzae following protein enrichment by heparin chromatography. Electrophoresis 18, 1193-1202.

Fountoulakis, M, Takacs, M. F., Berndt, P., Langen, H., Takacs, B., 1999. Enrichment of low abundance proteins of Escherichia coli by hydroxyapatite chromatography. Electrophoresis 20,2181-2195.

Glupczynski, Y., 1998. Microbiological and serological diagnostic tests for Helicobacter pylori: an overview. British Medical Bulletin (Eds Farthing, M. J. G., Patchett, S. E.) 54, 175-186.

Heukeshoven, J., Dernick, R, 1988. Improved silver staining procedure for fast staining in PhastSystem Development Unit I. Staining of sodium dodecyl sulfate gels. Electrophoresis 9, 28-32.

Jungblut, P. R, Bumann, D., Haas, G., Zimny-Arndt, U., Holland, P., Lamer, S., Siejak, F., Aebischer, A., Meyer, T. F. , 2000. Comparative proteome analysis of Helicobacter pylori. Mol. Microbiol. 36, 710-725.

Karlsson, K., Cairns, N., Lubec, G., Fountoulakis, M., 1999. Enrichment of human brain proteins by heparin chromatography. Electrophoresis 20, 2970-2976.

Kimmel, B., Bosserhoff, A., Frank, R., Gross, R., Goebel, W., Beier, D., 2000. Identification of immunodominant antigens from Helicobacter pylori and evaluation of their reactivities with sera from patients with different gastroduodenal pathologies. Infect. Immun. 68, 915-920.

Laemmli, U. K, 1970. Cleavage of structural proteins during the assembly of the head of bacteriophiage T4. Nature. 227, 680-685.

Lelwala-Guruge, J., Schalén, C., Nilsson, I., Ljungh, A., Tyszkiewicz, T., Wikander, M., Wadström, T., 1990. Detection of antibodies to Helicobacter pylori cell surface antigens. Scand. J. Infect. Dis. 22, 457-465.

Marshall, B., 1994. Helicobacter pylori. Am. J. Gastroenterol. 89 [suppl], S116-5128.

McAtee, C. P., Lim, M. Y., Fung, K., Velligan, M., Fry, K., Chow, T. P., Berg, D. E., 1998 a. Characterization of a Helicobacter pylori vaccine candidate by proteome techniques. J. Chromatogr. B Biomed. Sci. Appl. 714, 325-33.

McAtee, C. P., Lim, M. Y., Fung, K, Velligan, M., Fry K, Chow, T. P., Berg, D. E., 1998 b. Identification of potential diagnostic and vaccine candidates of Helicobacter pylori by two-dimensional gel-electrophoresis, sequence analysis, and serum profiling. Clin. Diagn. Lab. Immunol. 5, 537-542.

Meijer, B. C., Thijs, J. C., Kleibeuker, J. H., van Zwet, A. A., Berrelkamp R. J. 1997. Evaluation of eight enzyme immunoassays for detection of immunoglobulin G against Helicobacter pylori. J. Clin. Microbiol. 35, 292-294.

Nilsson, I., Ljungh, A., Aleljung, P., Wadstrom, T., 1997. Immunoblot assay for serodiagnosis of Helicobacter pylori infections. J. Clin. Microbiol. 35, 427-432.

Nilsson, I., Utt, M., Nilsson, H. O., Ljungh, A., Wadstrom, T. 2000.Two-dimensional electrophoretic and immunoblot analysis of cell surface proteins of spiral-shaped and coccoid forms of Helicobacter pylori. Electrophoresis 21, 2670-2677.

Soltesz, V., Zeeberg, B., Wadström, T., 1992. Optimal survival of Helicobacter pylori under various transport conditions. J. Clin. Microbiol. 30, 1453-1456.

Tomb, J-F, White, O., Kerlavage, A. R., Clayton, R. A., Sutton, G. G., Fleischmann, R. D., Ketchum, K. A., Klenk, H. P., Gill, S., Dougherty, B. A., Nelson, K., Quackenbush, J., Zhou, L., Kirkness, E. F., Peterson, S., Loftus, B., Richardson, D., Dodson, R. Khalak, H. G., Glodek, A., McKenney, K., Fitzegerald, L. M., Lee, N., Adams, M. D., Hickey, E. K, Berg, D. E., Gocayne, J. D., Utterback, T. R., Peterson, J. D., Kelley, J. M., Cotton, M. D., Weidman, J. M., Fujii, C., Bowman, C., Watthey, L., Wallin, E., Hayes, W. S., Borodovsky, M., Karp, P. D., Smith, H. O., Fraser, C. M., Venter, J. C., 1997. The complete genome sequence of the gastric pathogen Helicobacter pylori. Nature, 388, 539-547.

Wang, J-T., Chang, C-S., Lee, C-Z., Yang, J-C., Lin, J-T., Wang, T-H., 1998.Antibody to a Helicobacter pylori species specific antigen in patients with adenocarcinoma of the stomach. Biochem. Biophys. Res. Commun. 244,360-363.

Yamaoka, Y., Kodama, T., Graham, D. Y., Kashima, K, 1998. Search for putative virulence factors of Helicobacter pylori: the low-molecular-weight (33-35 K) antigen. Dig. Dis. Sci. 43,1482-1487.

TABLE 1

Immunogenic AGE-HepBP proteins of H. pylori strain 17874 identified from 2-DE gel by MS/MS sequencing

| | | | | |
|---|---|---|---|---|
| Spot | M_{I}s in kDa | pI | MS/MS sequence | Protein name |
| number^{a)} | (experimental) | (experimental) | (internal peptide) | (TIGR) |

| | | | | | | |
|---|---|---|---|---|---|---|
| 1 | 30 | 9.34 | TTDSSAGVLATVDGR | SEQ ID NO:1 | Cell binding factor 2 | HP0175 |
| 2 | 30 | 9.24 | DSPVTYTYEQAK | SEQ ID NO:2 | Cell binding factor 2 | HP0175 |
| 3 | 26 | 8,67 | LFGFNALVDR | SEQ ID NO:3 | Urease A | HP0073 |
| 4 | 26 | 8.01 | LFGFNALVDR | SEQ ID NO:3 | Urease A | HP0073 |
| 5 | 26 | 9.11-9.33 | MVVVGWLGVNSAK | SEQ ID NO:4 | Hypothetical protein | HP0231 |
| 6 | 25 | 9.04 | DPSNLYATEFDLVK | SEQ ID NO:5 | Outer membrane protein | HP1564 |

^{a)}Corresponds to the numbers in FIG. 3A, B, C

1. A method of identifying immunogenic Helicobacter pylori-specific surface proteins binding specifically to polysulphated molecules, comprising the steps of
cultivating H. pylori bacteria in vitro,
isolating the cultivated bacteria,
releasing the basic surface proteins by acid glycine extraction,
removing the bacteria
purifying the glycine extract to produce a Helicobacter pylori-specific surface protein product, and
subjecting the protein product to a two-dimensional immunoblot to identify immunogenic proteins.

2. The method according to claim 1, wherein
the cultivating in vitro is performed in an agar medium or broth,
the isolation of the bacteria is performed by collecting the grown cells into a phosphate buffered saline (PBS) and centrifugation to produce a pellet,
releasing the basic surface proteins from the pellet by acid glycine extraction,
removing the bacteria by centrifugation, and
purifying the glycine extract by diluting the supernatant from the centrifugation with PBS, adjusting the pH to 6.5 and subjecting the mixture to heparin affinity chromatography to produce the Helicobacter pylori-specific surface protein product.

3. A Helicobacter pylori-specific surface protein product binding specifically to polysulphated molecules, which is obtainable by
cultivating H. pylori bacteria in vitro,
isolating the cultivated bacteria,
releasing the basic surface proteins by acid glycine extraction,
removing the bacteria, and
purifying the glycine extract to produce the Helicobacter pylori-specific surface protein product.

4. The protein product according to claim 3, wherein
the cultivating in vitro is performed in an agar medium or broth,
the isolation of the bacteria is performed by collecting the grown cells into a phosphate
buffered saline (PBS) and centrifugation to produce a pellet,
releasing the basic surface proteins from the pellet by acid glycine extraction,
removing the bacteria by centrifugation, and
purifying the glycine extract by diluting the supernatant from the centrifugation with PBS, adjusting the pH to 6.5 and subjecting the mixture to heparin affinity chromatography to produce the Helicobacter pylori-specific surface protein product.

5. The protein product according to claim 3 or 4, wherein the product comprises at least one protein selected from the group consisting of
a) a protein having a molecular weight (MW) of 30 (31.3) kDa, an isoelectric point (pI) of 9.3 (9.1) and comprising the amino acid sequence SEQ ID NO: 1 and/or SEQ D NO: 2,
b) a protein having a MW of 26 (27.7), a pI of 9.1-9.3 (9.0). and comprising the amino acid sequence SEQ ID NO: 4, and
c) a protein having a MW of 25 (28.7). a pI of 9.0 (8.8) and comprising the amino acid sequence SEQ ID NO: 5.

6. A Helicobacter pylori-specific surface protein binding specifically to polysulphated molecules and having a MW of 30 (31.3) kDa, a (pI) of 9.3; (9.1) and comprising the amino acid sequence SEQ ID NO: 1 and/or SEQ ID NO: 2.

7. A Helicobacter pylori-specific surface protein binding specifically to polysulphated molecules and having a MW of 26 (27.7), a pI of 9.1-9.3 (9.0) and comprising the amino acid sequence SEQ ID NO: 4.

8. A Helicobacter pylori-specific surface protein binding specifically to polysulphated molecules and having a MW of 25 (28.7), a pI of 9.0 (8.8) and comprising the amino acid sequence SEQ ID NO: 5.

9. Use of a protein product according to any one of claims 3-5 or a protein according to any one of claims 6-8 as a diagnostic antigen in all immunoassay.

10. Use of a protein product according to any one of claims 3-5 or a protein according to any one of claims 6-8 as an immunizing component in a vaccine against H. pylori infection.

11. An immunoassay for the determination of the presence of H. pylori bacteria in a biological sample from a human patient, wherein a protein product according to any one of claims 3-5 or a protein according to any one of claims 6-8 is used as a diagnostic antigen.

12. A vaccine composition against a H. pylori infection in a human patient comprising as an immunizing component at least one protein product according to any one of claims 3-5 or a protein according to any one of claims 6-8, together with a pharmaceutically acceptable vehicle.
subjecting the protein product to a two-dimensional immunoblot to identify immunogenic proteins.


SEQUENCE LISTING (5 sequences)

SEQ ID NO: 1 - protein, 15 aa, Helicobacter pylori
TTDSSAGVLATVDGR

SEQ ID NO: 2 - protein, 12 aa, Helicobacter pylori
DSPVTYTYEQAK

SEQ ID NO: 3 - protein, 10 aa, Helicobacter pylori
LFGFNALVDR

SEQ ID NO: 4 - protein, 13 aa, Helicobacter pylori
MVVVGWLGVNSAK

SEQ ID NO: 5 - protein, 14 aa, Helicobacter pylori
DPSNLYATEFDLVK